(19) United States
(12) Patent Application Publication
(10) Pub. No.: US 2003/0133993 A1
(43) Pub. Date: Jul. 17, 2003

(54) Novel synergistic antimicrobial compositions and methods

(75) Inventors: Alex Hutcheson, Dublin, CA (US); Christopher Hawk, Mill Creek, WA (US); Frank Swenson, Alameda, CA (US)

Correspondence Address: QUINE INTELLECTUAL PROPERTY LAW GROUP, P.C., P O BOX 458, ALAMEDA, CA, 94501, US

(73) Assignee: SureCide Technologies, LLC, Alameda, CA (US)

(21) Appl. No.: 10/188,020

(22) Filed: Jul. 1, 2002

Related U.S. Application Data
(60) Provisional application No. 60/302,815, filed on Jul. 3, 2001.
(60) Provisional application No. 60/302,950, filed on Jul. 2, 2001.

(51) Int. Cl. A61K 33/26; A01K 29/00

(52) U.S. Cl. 424/647; 514/55; 119/172; 119/173

(57) ABSTRACT

The present invention provides antimicrobial compositions and methods for reducing the number and/or the viability of microbial populations on surfaces exposed to animal feces, such as animal litter and animal containment areas.

### CROSS-REFERENCE TO RELATED APPLICATIONS

This application claims priority to, and benefit of U.S. provisional patent applications U.S. Ser. No. 60/302,950 filed Jul. 2, 2001 and U.S. Ser. No. 60/302,815 filed Jul. 3, 2001, pursuant to 35 U.S.C. §119(e) and any other applicable statute or rule, which applications are incorporated by reference herein.

### FIELD OF THE INVENTION

This invention relates to the control and/or mitigation of microbial contamination in or around animal containment areas and animal litter.

### BACKGROUND OF THE INVENTION

Food-borne microbial infections represent a constant and grave threat to human health. Chief pathogens among the bacterial genera responsible for outbreaks of food poisoning include, but are not limited to, Escherichia, Salmonella, Shigella, Campylobacter, and Listeria. Infections may be caused by microbes originating from a number of sources, including, but not limited to, fecal matter present on the animal prior to slaughter. Containment of animals in cages, holding pens or stalls increases the duration of exposure of the animals to fecal matter present in the environment. The extended exposure to pathogen allows for a greater extent of microbial attachment or contamination, and increases the possibility that any pathogenic organisms are transferred either via direct contact with the animal, or by contamination of a resulting food products (e.g., during the slaughter process).

For example, in the poultry industry, contamination of raw poultry product by pathogens by Salmonella is found in approximately 1 out of 4 birds processed and sold as raw product in the United States. The incidence numbers are higher for Escherichia (for example, E. coli O157:H7) and Campylobacter. Food poisoning associated with contaminated raw poultry product is a serious concern, and methods for reducing contamination at the factory are a priority for poultry producers and public oversight agencies (e.g., USDA, FDA).

In a nationwide study by the USDA, 11% of sampled chicken litter was found to harbor viable Salmonella. It is likely that this contaminated material is a significant source for cross contamination in poultry flocks. There is a need in the art for methods and compositions which can be used to decrease the viability of pathogens associated with animal litter and the areas in which it is used (e.g., the cages, coops, or other containment areas). The present invention addresses this need by providing antimicrobial compositions and methods.

### SUMMARY OF THE INVENTION

The present invention provides antimicrobial compositions and methods for reducing the number and/or the viability of microbial populations on surfaces exposed to animal feces, such as animal litter and animal containment areas.

Treatment of materials regularly exposed to animal feces (animal litter, as well as cages, coops, stables, and other animal containment areas) with the compositions of the present invention reduces and/or inhibits the growth of harmful microorganisms present in the litter or containment area. As a result, the compositions and methods of the present invention also reduce the amount or extent of cross-contamination among animals prior to slaughter, a problematic issue that arises particularly during poultry processing. Furthermore, these treatments may have the added benefit of inhibiting mold, fungus, and other organisms already present on the litter, thereby reducing or prevent contamination of the litter during storage.

Accordingly, the present invention provides synergistic antimicrobial composition for treating a quantity of animal litter. The compositions include, but are not limited to, a preparation of at least one iron salt; a citrate composition; and a chitosan preparation. In a preferred embodiment, the antimicrobial composition comprises FeCl₃, citric acid, and a low molecular weight chitosan.

A range of concentrations of treatment components are considered in the methods and compositions of the present invention. For example, the concentrations of FeCl₃ and citrate can independently range from about 1 mM to about 100 mM, and optionally from about 1 mM to about 50 mM, or about 50 mM to about 100 mM. In addition, the range of concentrations of chitosan can vary from as low as about 0.001% to about 0.05% or about 0.1%. One of skill in the art will appreciate that the concentration of stress inducer employed in the methods will vary, depending upon, for example, the molecular weight ranges of the stress inducer employed, the nature of the material (e.g., the animal litter) being treated, and the microbial organisms to be affected. The relative amounts of iron salt and stress inducer can easily be determined empirically, using techniques known in the art.

In some embodiments, the composition is provided as a solid preparation, for example, in the form of a powder. Typically, the formulation is prepared as a 1:1 molar ratio of iron salt and citrate composition, to which between about 0.001% and about 1% chitosan is added. Optionally, the solid preparation includes about 0.01% chitosan or 0.1% chitosan. In other embodiments, the composition is prepared as a liquid, such as an aqueous solution (for example, a solution of about 100 mM FeCl₃, about 100 mM citrate, and about 0.1% chitosan, or a solution of about 50 mM FeCl3, about 50 mM citrate, and about 0.05% chitosan). Optionally, FeCl₂ can be used in place of FeCl₃ in either the solid or liquid preparations

The present invention also provides methods for preparing antimicrobial animal litter, as well as the treated litter product prepared by the methods. The methods of the present invention include the steps of a) providing a first quantity of animal litter; b) providing an antimicrobial treatment composition comprising at least one iron salt; a citrate composition; and a chitosan preparation; and c) exposing the first quantity of animal litter to the composition, thereby preparing an antimicrobial animal litter. Any number of materials can be employed as the animal liter to receive treatment, such as rice hulls, straw, corn husks, clay, diatomaceous earth; sawdust; wood chips, wood shavings, recycled paper products; agricultural waste materials, gravel (or combinations thereof). The antimicrobial composition can be provided as a solid treatment composition (e.g., in powdered form), and used to coat or dust the animal litter. Alternatively, the antimicrobial composition is provided in liquid form, and the animal litter is exposed to the composition by soaking, spraying, and the like.

The solid or liquid forms of the treatment composition can be prepared by any of a number of standard techniques known to one of skill in the art. In a preferred embodiment for preparing a liquid composition for use in the methods of the present invention, the chitosan component is prepared in an organic acid (e.g., acetic acid) prior to combination with the iron salt and citrate components. Optionally, the methods include the additional step of removing an unabsorbed portion of the treatment solution from the animal litter.

In certain embodiments of the methods for preparing an antimicrobial animal litter, the treated litter is combined with a portion of untreated litter, thereby extending the antimicrobial action of the litter. For example, the treated litter can be combine with up to an equal portion of untreated litter without substantial loss of antimicrobial action.

The present invention further provides methods for reducing the microbial population in an animal containment area, as well as methods for increasing the storage life of an animal litter using the compositions as described herein.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification and the appended claims, the singular forms “a”, “an”and “the” include plural referents unless the content clearly dictates otherwise. Thus, for example, reference to “a surface” includes a combination of two or more surfaces; reference to “a microbe” includes mixtures of microbes, and the like.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

The terms “litter” and “animal litter” are interchangeably used herein to refer to material used in cages, stalls, or other containment areas for bedding, insulation, or absorption of urine/feces.

As used herein, the term “microbe” refers to both pathogenic and nonpathogenic, typically unicellular organisms, including, but not limited to, bacteria, yeast, molds and fungal cells.

The term “antimicrobial” as used herein refers to a compound, treatment, or effect that is biocidal (e.g., kills cells or components of cells), biostatic (e.g., prevents further growth of cells), or a combination thereof. As such, a classification as an “antimicrobial compounds” is meant to encompass, but is not limited to, compounds having bacteriostatic, bactericidal, fungistatic, fungicidal, antiparasitic and/or antiviral activity.

The term “chitosan” as used herein refers to a polycationic polymer of (β1,4)-linked glucosamine residues, wherein at least about 50% (or optionally, about 70%, about 80%, about 90%, about 95% or about 99%) of the amino groups at the C-2 position are present as free amines (e.g., not acetylated).

The term “citrate” as used herein refers to both the acid form (citric acid) as well as the salt form (sodium citrate, ammonium citrate, and the like).

The term “organic acids” refer to any of a number of carbon-containing acid compounds, such as acetic, citric, tartaric, or mandelic acid.

As used herein, the term “about” refers to the variance in a numerical value, typically referring to +/−10% of the stated value.

### DETAILED DESCRIPTION

The present invention provides antimicrobial compositions and methods for reducing the number and/or the viability of microbial populations on surfaces exposed to animal feces, such as animal litter and animal containment areas.

While the methods and compositions are applicable for reducing microbial exposure of any number of animals (including pets as well as livestock), the present invention is particularly useful for use in conjunction with poultry processing. Cross contamination of bird carcasses at various stages of processing is an important factor in the final levels of contamination of raw product. Of particular concern are the “scalder” and “chiller” process points, where all of the birds are exposed to common water baths with potentially high levels of bacterial contamination. One way to reduce the microbial contamination in poultry processing facilities is to reduce the number of contaminated birds going into the plant. Cross-contamination of birds can arise, for example, from exposure bacteria in their shared litter. Treatment of the litter to reduce and/or inhibit the growth of harmful organisms such as Salmonella would reduce the microbial load entering the poultry processing plant.

Antimicrobial Composition

The present invention provides antimicrobial compositions for treating a quantity of animal litter, such as chicken litter. The antimicrobial compositions include, but are not limited to, a preparation of at least one transition metal salt and two or more microbial stress inducers. In a preferred embodiment, the antimicrobial composition includes at least one iron salt, a citrate composition and a chitosan preparation. These components act in a synergistic manner to reduce the microbial load present on the treated surface (e.g., the litter or the containment area).

A number of microbial populations commonly found on poultry and other food products can be targeted using the composition and methods of the present invention, including, but not limited to, gram negative bacteria such as Escherichia, Salmonella, Shigella (and other Enterobacteracae), Vibrio (e.g., Vibrio cholerae), Streptococci, Campylobacter, and Pseudomonas; gram-positive bacteria such as Staphylococci, Listeria, Neisseria, and Klebsiella; and Anaerobes, including but not limited to Bacteroides and Clostridium. The compositions and methods of the present invention are effective in reducing the viability of these and other microbial species in animal litter and/or animal containment areas, including those listed in PCT publication WO 01/08143, filed on Mar. 12, 2001.

Iron Salts

A number of iron salts can be employed in the present invention. For example, various salts or organic complexes of iron, such as acetate, ammonium citrate, ammonium oxalate, ammonium sulfate, bromide, chloride, citrate, fluoride, fumarate, hydroxide, iodide, nitrate, oxide, phosphate, pyrophosphate, sulfate, and tartrate, can be employed in the compositions and methods of the present invention. In a preferred embodiment, the iron salt used in the antimicrobial composition is FeCl₃ (ferric chloride).

Optionally, various salts or organic complexes of other transition metals may be used in combination with, or instead of, the iron salts. The transition metals include, but are not limited to, the elements chromium, manganese, iron, nickel, copper, zinc, and molybdenum. Further details regarding the antimicrobial action of transition metal compositions can be found in, for example, US publication 02-0015697-A1 and PCT publication WO01/08143, both filed on Mar. 12, 2001 by Beckman (“Use of Enteric Iron in the Decontamination of Livestock and Poultry Carcasses”), which references are incorporated herein by reference in their entirety.

In one embodiment of the present invention, the antimicrobial composition includes an aqueous treatment solution comprising about 100 mM FeCl₃. In another embodiment, the antimicrobial composition includes an aqueous treatment solution comprising about 50 mM FeCl₃. In a further embodiment, the iron salt is FeCl₂. Optionally, the iron salt is prepared in water or in hypo-osmotic saline solutions.

Citrate Compositions

In addition to the iron salt, the composition includes at least two microbial stress inducers. “Stress inducers” are compounds that induce a stress response in a microbial biological system, or otherwise alter the biological functioning of a cell or organism. Surprisingly, it was found in the present invention that the stress inducers citrate and chitosan act synergistically with the iron salt, thereby enhancing the microbiocidal activity of the compositions of the present invention.

A number of citrate compositions can be used in the present invention, including, but not limited to, citric acid, sodium citrate, potassium citrate, ammonium citrate, magnesium citrate, calcium citrate, and the like. In one embodiment of the present invention, the antimicrobial composition includes an aqueous treatment solution comprising about 100 mM citrate. In another embodiment, the antimicrobial composition comprises about 50 mM citrate. Optionally, the citrate composition is also prepared in water, or in hypo-osmotic saline solutions (either separately from or concomitant with the iron salt).

Chitosan Preparations

It was an unanticipated discovery that the chitosan component of the antimicrobial formulation acted in a synergistic manner with the iron salt and citrate, to enhance the biocidal activity of the composition. Chitosan is a positively charged amino-polysaccharide composed primarily of β(1,4)-linked D-glucosamine residues. Two forms of chitosan are generally available, the free amine (—NH2) version and the cationic (—NH3+) version. The free amine chitosan is soluble in acidic solutions, less soluble at pH values greater than about 6.5, and typically insoluble in most organic solvents. In contrast, the cationic form of chitosan is soluble at pH values greater than about 6.5. However, cationic chitosan forms fairly viscous solutions, and will form gels in the presence of polyanions. Either form of chitosan can be used in the compositions and methods of the present invention.

Commercial forms of chitosan are typically prepared by deacetylation of chitin, the β(1,4)-linked N-acetyl D-glucosamine polymer that is the major component of arthropod exoskeletons. Thus, chitosan preparations are typically non-homogeneous polymers containing both deacetylated glucosamine, as well as some remaining N-acetylated glucosamine residues (between about 1-5% for pharmaceutical grade chitosan, to as much as 10-30% for alternative grades of the product). Furthermore, the molecular weight of the polycationic chitosan polymer can vary from 25-50 kDa (for low molecular weight forms) to 500-1000 kDa or greater (high molecular weight chitosan). The molecular weight of the chitosan product will depend in part upon the source of the chitin “precursor” material and the methodology used (chemical vs. enzymatic). Chitosan derived from a variety of sources, including crab, shrimp, lobster and other arthropod exoskeletons, can be employed in the methods and compositions of the present invention, as can the low, medium, and high molecular weight compositions. Further details with respect to the use of chitosan as an antimicrobial agent can be found in, for example, U.S. Ser. No. 10/126,482 filed on Apr. 19, 2002 by Hawk et al. (“Methods and Compositions for Inhibiting Bacterial Attachment to Surfaces”), which reference is incorporated herein by reference in its entirety.

Chitosan preparations for use in the methods of the present invention optionally range from between about 0.001% and about 1% chitosan, and preferably between about 0.001% and about 0.1%. In one embodiment of the present invention, the chitosan concentration is about 0.1% (w/w), while in an alternate embodiment, the concentration of the chitosan preparation is about 0.05% (w/w). Optionally, the chitosan solution is prepared in one or more organic acids, including, but not limited to, acetic acid, lactic acid, citric acid, or a combination thereof. Alternatively, the chitosan can be prepared in an aqueous solution of water, propanol, isopropanol, ethanol, butylene glycol, or glycerin, or in a polar aprotic liquid such as DMSO. Optionally, the chitosan solution is prepared prior to combination with the iron salt and citrate components of the antimicrobial composition.

In some embodiments, the antimicrobial composition is a liquid composition. In other embodiments, the antimicrobial composition is provided in a solid form. Typically, the solid formulations of the present invention are prepared as a 1:1 molar ratio of iron salt and citrate composition, to which between about 0.001% and about 1% chitosan is added. Optionally, the solid preparation includes about 0.01% chitosan or 0.05% chitosan. The solid form can be used directly to treat the animal litter or containment area, for example, in the form of a powder, or it can be dissolved or suspended in a liquid prior to use.

Iodophores

In another embodiment of the present invention, the antimicrobial composition further includes an iodophore or other halide composition. One exemplary iodophore composition for use in the present invention is povidone-iodine complex. Alternatively, the halide composition is provided as a chitosan-halide preparation, such as trimethylammonium glycol chitosan iodide. Additional halides including, but not limited to, chloride, fluoride and bromide can also be employed.

Methods for Preparing Antimicrobial Animal Litters

Animal Litter Compositions

The present invention also provides methods for preparing antimicrobial animal litter. In a preferred embodiment, the animal litter comprises components suitable for use as chicken litter. Any number of litter components can be treated with the antimicrobial compositions of the present invention. Exemplary litter components include, but are not limited to, various agricultural waste materials or plant materials such as rice hulls, straw, and corn husks; gravel, clay and diatomaceous earth; fibrous peat; sawdust, wood chips, wood shavings, and other wood products; and recycled paper products such as shredded newspaper and cardboard. The animal litter can comprise an individual component as listed above, or combinations of components. One or more of these components can be treated with the antimicrobial composition, as described herein.

Treatment Schemes

In order to imbue the animal litter with antimicrobial properties, a quantity of the litter is exposed to the antimicrobial composition. However, the entire supply of animal litter need not be treated with the composition. A portion, or first quantity, of animal litter can be treated with the composition, then combined with a second portion or quantity of untreated litter. In one embodiment of the methods of the present invention, the treated and untreated portions are like, or similar, compositions of animal litter (e.g., both portions are rice hulls). In an alternate embodiment, the first quantity of animal litter to be treated with the antimicrobial composition can be a first component (e.g., rice hulls), while the second quantity can be a separate component (e.g., a recycled paper product), which when combined with the first component makes up the animal litter composition.

A number of ratios of treated and untreated litter compositions can be employed in the preparation of the litter composition while retaining the antimicrobial properties of the treated portion of litter. For example, the final litter composition can contain 90%, 75%, 60% or 50% treated litter while retaining the desired antimicrobial properties. Additionally, even lower percentages of treated animal litter are contemplated in the present invention (e.g., 45%, 35% or 25% treated litter component).

The quantity of animal litter to be treated can be exposed to the antimicrobial composition in a number of manners. The exposure method (e.g., method of dispersal of the antimicrobial composition within the litter component) is based in part upon the type of litter selected for treatment. Effective treatment schemes are easily determined by one of skill in the art would.

Any of the compositions of the present invention (as described previously) can be used to treat the animal litter in this manner. Typically, the antimicrobial composition is provided in either a solid or a liquid form. Thus, in one embodiment of the present invention, the animal litter is exposed to a solid formulation of the antimicrobial composition by providing a powdered form of the composition, and coating or dusting the animal litter with the composition. For example, approximately 200-450 g of antimicrobial composition can be prepared in powdered form and used to treat a cubic yard of animal litter.

In a preferred embodiment, the antimicrobial composition is provided in a liquid, suspended, or solution form. Optionally the solution is an aqueous solution, although amphiphilic solvents such as DMSO are also considered. The quantity of litter to be treated can be exposed to the antimicrobial composition by soaking the litter in the composition. Alternatively, the litter is sprayed with the antimicrobial composition. Optionally, after exposed to the liquid antimicrobial composition, the excess or unabsorbed portion is removed prior to drying the litter.

In one embodiment, the liquid composition used to treat the animal litter comprises an aqueous treatment solution of about 100 mM FeCl₃, about 100 mM citrate, and between about 0.001% and about 1% chitosan. In another embodiment, the antimicrobial composition comprises 50 mM FeCl₃, about 50 mM citrate, and between about 0.001% and about 1% chitosan. In other embodiments of the present invention, FeCl₂ is used as the iron salt; in these embodiments, the antimicrobial composition comprises, for example, 100 mM FeCl₂, about 100 mM citrate, and between about 0.001% and about 1% chitosan, or. In another FeCl₂ embodiment, the antimicrobial composition comprises 50 mM FeCl₂, about 50 mM citrate, and between about 0.001% and about 1% chitosan.

Optionally, the chitosan component of the composition is between about 0.001% and about 0.1% chitosan, or about 0.05%, or about 0.1% chitosan. In preparing the antimicrobial composition, the chitosan component is optionally suspended in an organic acid prior to combination with the FeCl₃ and citrate components. Exemplary organic acids which can be used for suspending the chitosan include, but are not limited to, citric acid, acetic acid, lactic acid, or combinations thereof.

The optimal duration of contact between the antimicrobial composition and the animal litter will depend in part upon the litter component being treated. Treatment times can range from as short as a few seconds, to longer periods of time, such as 10 minutes, 30 minutes, an hour, or several hours. The optimal concentration of treatment composition and length of time that the litter material should be exposed to the treatment preparation can be determined empirically by one of skill in the art, using, for example, methods known to those of skill in the art for evaluating microbiocidal activity. Typically, the surface is brought into contact with the antimicrobial composition for about 30 minutes. However, both shorter as well as longer contact durations are also acceptable. Alternatively, in some embodiments (e.g., when using a powdered form of the composition), the antimicrobial composition can optionally be left on the treated litter.

Furthermore, the microbial species (or groups of species) to be targeted can play a role in determining the treatment parameters. Optimal treatment conditions can easily be determined, using standard methods for evaluating microbiocidal activity commonly known to one of skill in the art.

Methods for Reducing Microbial Populations

The present invention also provides methods method for reducing the microbial population in an animal containment area. The animal containment area can be an indoor or outdoor structure, including, but not limited to, cages, coops, sheds, stables, gated areas and the like. In one embodiment of these methods, the animal containment area is supplied with the antimicrobial litter composition of the present invention. Optionally, the animal containment area is regularly re-supplied with antimicrobial litter (e.g., between flocks of poultry) In one embodiment of the methods of the present invention, an application of litter is used for 5-6 consecutive hatches, with a fresh layer of litter applied between flocks. In another embodiment, the animal containment area itself is treated with the antimicrobial litter composition, for example, by spraying a liquid or powdered form of the antimicrobial composition.

Furthermore, the present invention includes methods for increasing the storage life of an animal litter. The method includes treating the animal litter with the antimicrobial composition, thereby reducing microbial spoilage and/or contamination of the litter.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. Thus, the following examples are offered to illustrate, but not to limit the claimed invention.

For the following examples, a culture of Salmonella choleraesuis (strain 10708) was grown for 24 hours at 37° C. In the following examples, each of the treatment compositions were tested using rice hulls as the animal litter. After exposing the litter to the antimicrobial composition, the preparations were filtered and the impregnated litter filtrates were dried and placed in separate culture tubes. Sterile nutrient broth (NB) was added to each tube and appropriate tubes were inoculated with 10³ cfu/mL of Salmonella. All tubes were incubated overnight and examined visually. Visual interpretation results were confirmed by subculturing all culture tubes to plated media.

### Example 1

A quantity of litter (approximately 5 g of rice hulls) was soaked for 30 minutes in 100 mL of one of the following aqueous test preparations:

a) 100 mM FeCl₃+100 mM citrate

b) 0.1% chitosan (ChitoClear fg 95 from Primex Ingredients ASA, Norway)

c) 100 mM FeCl₃+100 mM citrate+0.1% chitosan

d) ddH₂O (control)

After soaking, the test preparations were removed from the litter material by vacuum filtration. The wet litter was spread on paper towels and allowed to dry between about 5 hours and overnight, followed by approximately 45 minutes exposure to desiccant.

Each of the treated litter samples was tested in triplicate for the ability to reduce or inhibit growth of an inoculated Salmonella culture. Three aliquots of litter (0.5 g each) from each of the four batches of treated litter (FeCl₃/citrate, chitosan, FeCl₃/citrate/chitosan, and water) were measured and placed in test tubes, to which 18 mL of microbial nutrient broth (Hardy Diagnostics, Santa Maria Calif.) was added. Control test tubes containing untreated litter (e) and no litter (f) were also prepared.

The tubes containing the treated litter were inoculated with 2 mL of a 10⁴ cells/mL stock Salmonella solution (final concentration of 10³ cells/mL). The inoculated tubes were incubated at 37° C. for 24 hours, then visually examined for positive cell growth. In addition, a sample from each test tube was subcultured onto nutrient and MacConkey agar. The results from these tests are shown in Tables 1 and 2.

The experiment was performed in triplicate; all three replicates of each test showed identical results. The litter treated with the iron-containing compositions (a and c) was darker in color than the other litters. The chitosan-treated litter (b) and the control litter (d) looked identical to the litter from an external source. However, the litter treated with chitosan (b and c) released a flaky precipitate when the growth media was added. This eventually settled out and/or went back into solution. As a side note, the control with no cells (e) grew a fungus that formed a layer on top of the growth media.

Litter treated with 100 mM ferric chloride and 100 mM citrate inhibited the growth of Salmonella choleraesuis (and the strains already present on the litter) when tested in liquid media. Though this litter appears to be largely bactericidal, subculture results indicated that there was some bacteriostatic activity. Though the number of viable cells was dramatically reduced from the controls, a few colonies appeared when the liquid from the tubes was streaked onto solid media. These colonies appeared to be Salmonella. However, even after several days of incubation, the cultures did not grow to confluence. Apparently, the few remaining cells can not grow in the presence of the treated litter.

Litter treated with chitosan alone did not inhibit the growth of any of the strains. However, the addition of chitosan to the iron/citrate treatments dramatically improved the efficacy of the litter, suggesting a synergistic effect among the treatment components. When the litter was treated with all three compounds, no Gram negative strains grew in the liquid media. The subcultures showed no Gram negative colonies and one Gram positive strain. Presumably, this Gram positive strain was allowed to grow in the absence of the Gram negatives, which may have competitively inhibited its growth in other tests. Again, the litter appeared to have bacteriostatic effects on this strain, as it did not grow to confluence after several days of incubation.

Treatment of animal litter with the compositions of the present invention inhibits the growth of harmful microorganisms introduced to the litter. Optionally, this will reduce the amount of cross-contamination among birds before slaughter. Furthermore, these treatments may have the added benefit of inhibiting mold, fungus, and other organisms already present on the litter. This may prevent spoilage and contamination of the litter during storage.

TABLE 1
Growth of Salmonella upon Treated Litter versus Untreated Litter

| a | b | c | d | e | f |
|---|---|---|---|---|---|
| FeCl₃ + citrate | chitosan | FeCl₃ + citrate + | dH₂O treated | untreated | no litter |
| treated litter | treated litter | chitosan treated | litter | litter | (− control) |
| | | litter | (+ control) | (− control) | |
| 100 mM FeCl₃ | — | 100 mM FeCl₃ | — | — | — |
| 100 mM citrate | — | 100 mM citrate | — | — | — |
| — | 0.1% chitosan | 0.1% chitosan | — | — | — |
| cells | cells | cells | cells | no cells | no cells |
| No Growth | Growth | No Growth | Growth | Growth | No Growth |

TABLE 2
Subculture Results

| | | Nutrient Agar | MacConkey Agar |
|---|---|---|---|
| a | 100 mM Fe | One Gram negative strain that | One Gram negative strain that |
| | 100 mM Citrate | appears to be Salmonella. | appears to be Salmonella. |
| | | Numbers dramatically reduced | Numbers dramatically reduced |
| | | from control. Only a few colonies. | from control. Only a few |
| | | | colonies. |
| b | .1% | At least one Gram positive strain. | At least two different gram |
| | chitosan | Flat, fuzzy looking colonies. | negatives. One of which was |
| | | | Salmonella. |
| c | 100 mM Fe | One gram positive strain. Few | No Growth |
| | 100 mM Citrate | snowflake shaped fuzzy white | |
| | .1% chitosan | colonies. | |
| d | Control, With | Mixed, heavy growth. At least | Heavy growth. At least two |
| | Cells | two Gram negatives. One strain | different gram negatives. One of |
| | | was Salmonella. | which was Salmonella. Some |
| | | | pink colonies and some white |
| | | | colonies. |
| e | Control, No | Two Gram negatives. Some small | Two Gram negatives. Mostly |
| | Cells | yellow colonies and some white | large pink colonies with a few |
| | | colonies. | small brown colonies. |
| f | Control, No | No Growth | No Growth |
| | litter and No | | |
| | Cells | | |

### Example 2

Litter was prepared as described in Example 1. Prior to incubation with the Salmonella culture, quantities of the treated litter were mixed with untreated litter in the following ratios:

a) 0.5 g iron/citrate/chitosan litter (“undiluted”)

b) 0.25 g iron/citrate/chitosan litter+0.25 g untreated litter (1:1 ratio)

c) 0.1 g iron/citrate/chitosan litter+0.4 g untreated litter (1:4 ratio)

d) 0.5 g iron/citrate litter (“undiluted”)

e) 0.25 g iron/citrate+0.25 g untreated litter (1:1 ratio)

f) 0.1 g iron/citrate+0.4 g untreated litter (1:4 ratio)

The litter samples were aliquoted, inoculated and analyzed as described in Example 1. The results from these tests are shown in Table 3.

The assays were performed in duplicate; both replicates of each test showed identical results. As observed in Example 1, the litter samples representing the higher concentrations of ferric chloride (100 mM Fe+100 mM Citrate+0.1% chitosan and 100 mM Fe+100 mM Citrate) prevented growth under the test conditions. They inhibited both the Salmonella and the bacteria already present on the litter. The litter used in this experiment was prepared as for Example 1 and was stored at room temperature for a week before the experiment was performed. These results indicate that the litter retains its activity while in storage.

The 50% mixtures of both treatments (iron/citrate and iron/citrate/chitosan) also prevented growth. The 20% mixtures did not appear to be as effective under the conditions tested. Thus, mixtures of treated and untreated litter can be employed in the methods of the present invention. This would reduce the amount of litter that needed to be treated, thereby reducing the cost of implementing the methods of reducing microbial load in animal containment structures, such as poultry facilities.

TABLE 3
Growth of Salmonella upon Various Ratios of Treated Litter

| a | b | c | d | e | f |
|---|---|---|---|---|---|
| 100% | 50% | 20% | 100% | 50% | 20% |
| 100 mM Fe | 100 mM Fe | 50 mM Fe | 100 mM Fe | 100 mM Fe | 100 mM Fe |
| 100 mM Cit | 100 mM Cit | 50 mM Cit | 100 mM Cit | 100 mM Cit | 100 mM Cit |
| .1% Chitosan | .1% Chitosan | .1% Chitosan | | | |
| No Growth | No Growth | Growth | No Growth | No Growth | Growth |

### Example 3

The following test preparations were used to treat 7 g of litter using the protocol described in Example 1:

a) 50 mM FeCl₃+50 mM citrate+0.05% chitosan

b) 10 mM FeCl₃+10 mM citrate+0.01% chitosan

c) 1 mM FeCl₃+1 mM citrate+0.001% chitosan

d) 50 mM FeCl₃+50 mM citrate

e) 10 mM FeCl₃+10 mM citrate

f) 1 mM FeCl₃+1 mM citrate

The litter samples were aliquoted, inoculated and analyzed as described in Example 1. The experiment was performed in duplicate; both replicates of each test showed identical results. The results from these tests are shown in Table 4.

Litter treated with 50 mM iron was only slightly darker than the control. Lower concentrations showed very little or no color change.

The litter treated with 50 mM FeCl₃+50 mM citrate was effective against all strains present, both in the presence and in the absence of chitosan. Growth was observed in the 10 mM tubes, but visual inspection clearly showed that some inhibition had occurred. The 1 mM tubes were much more confluent than the 10 mM tubes.

Though the 10 mM iron treatments were not as effective as the higher concentrations, less microbial growth was observed in these tubes than in the 1 mM iron-containing tubes. Growth in the nutrient broth (NB) control tube (which was not inoculated) indicates that the culture medium was contaminated. Therefore, all treatments in this experiment were also tested on this contaminant. Since the litter is not sterile to begin with, this is probably not of much concern.

The results of this experiment show an interesting correlation with Example 2 where we found that a 50% mixture of untreated litter and litter treated with 100 mM Fe+100 mM citrate+0.1% chitosan was effective at reducing microbial growth. Litter sample (a) in Example 2 corresponds with the 100% litter treated with 50 mM Fe+50 mM Citrate+0.05% chitosan (sample a) in this experiment. The same total weight of litter (0.5 g) was used in the assays, confirming that the use of 50 mM treatment-litter is as effective as use of a 1:1 ratio of untreated and 100 mM-treated litter.

In the present example, the lowest effective treatment concentrations are between about 50% and about 10% of the concentrations used in Example 1. However, additional concentrations and different ratios of iron, citrate and chitosan are contemplated in the compositions and methods of the present invention.

TABLE 4
Growth of Salmonella upon Various Ratios of Treated Litter

| a | b | c | d | e | f | g | h |
|---|---|---|---|---|---|---|---|
| 50 mM Fe | 10 mM Fe | 1 mM Fe | 50 mM Fe | 10 mM Fe | 1 mM Fe | Growth | NB |
| 50 mM | 10 mM | 1 mM | 50 mM | 10 mM | 1 mM | Control | Control |
| citrate | citrate | citrate | citrate | citrate | citrate | | |
| 0.05% | .01% | .001% | | | | | |
| chitosan | chitosan | chitosan | | | | | |
| cells | cells | cells | cells | cells | cells | cells | no cells |
| No | Inhibited | Growth | No | Inhibited | Growth | Growth | Growth |
| Growth | Growth | | Growth | Growth | | | |

### Example 4

In this experiment, a range of treatment composition concentrations were examined in order to determine the lowest effective concentrations for producing growth inhibitory litter. In addition, the ferric chloride and citrate components were tested individually, to confirm that the observed biocidal/germicidal results were not due to only one component of the treatment mixture.

The following test preparations were used to treat 7 g of litter using the protocol described in Example 1:

a) HCl,pH 1.18

b) Acetic Acid (50 μl in 100 mL)

c) 50 mM FeCl₃+50 mM Citrate+0.05% Chitosan

d) 40 mM FeCl₃+40 mM Citrate+0.04% Chitosan

e) 30 mM FeCl₃+30 mM Citrate+0.03% Chitosan

f) 50 mM FeCl₃+50 mM Citrate

g) 40 mM FeCl₃+40 mM Citrate

h) 30 mM FeCl₃+30 mM Citrate

i) 50 mM FeCl₃

j) 40 mM FeCl₃

k) 30 mM FeCl₃

l) 50 mM Citrate

m) 40 mM Citrate

n) 30 mM Citrate

The litter samples were aliquoted, inoculated and analyzed as described in Example 1. The results from these tests are shown in Tables 5A, 5B and 5C.

As in the previous experiments, both replicates of each test showed identical results. The NB negative control did not grow (the growth media was not contaminated in this experiment). In addition, even at 50 mM Fe+50 mM citrate+0.05% chitosan, slight microbial growth occurred after incubation. Slightly more growth was seen in the 50 mM Fe+50 mM citrate (without chitosan) tubes. Tubes with only iron or only citrate showed heavy growth.

The samples treated with either 40 mM and 30 mM iron salt showed more growth than samples treated with 50 mM iron, but a similar growth gradient was observed (e.g., increasing growth from the mixture of all three components to iron+citrate without Chitosan to the individual components).

Unexpectedly, litter treated with just HCl or acetic acid was completely ineffective. The pH of the solutions in the tubes after incubation decreased with increasing treatment concentration (as expected). However, even at the highest treatment concentrations, the pH did not drop below 4.5.

Contrary to the results seen in Example 3, litter treated with 50 mM concentrations of iron and citrate in combination did not completely prevent growth. We suspect that this is the result of experimental error, since we have consistently observed this litter to be effective. Still, these treatments showed substantially less growth than the individual treatment components, which were totally ineffective. Since we are fairly confident that the growth in the 50 mM tubes is not reproducible, the results for the lower concentrations (which showed even more growth) are also in question. Thus, concentrations below 50 mM may optionally still be effective treatment concentrations.

At all concentrations tested, the addition of chitosan to the iron/citrate mixture resulted in slightly less growth. Since litter treated with chitosan alone is ineffective at reducing microbial populations, these results suggest that there is a synergistic interaction among these compounds. Ferric chloride and citrate also appear to operate synergistically with each other. Neither was effective as an individual litter treatment, but litter treated with both iron and citrate dramatically inhibited bacterial growth. This interaction has also been observed in other unpublished experiments (also see, for example, 60/292,886, filed May 22, 2001).

The litter treated with higher concentrations of iron, citrate and chitosan resulted in solutions of lower pH. However, none of the solutions were below pH 4.5. Though the acidic environment may have some inhibitory effect, it is clear that the germicidal results are not solely due to pH. Litter treated with iron alone resulted in a pH below 5, yet heavy growth was still observed.

In this experiment, we also treated litter with either HCl pH 1.18 or acetic acid (50 μl glacial acetic acid in 100 mL, equal to the highest concentration used in the chitosan solutions). Neither treated litter showed any inhibitory effects. The pH of the growth media after incubation in the presence of this litter was only slightly acidic (pH˜6).

TABLE 5A
Growth of Salmonella upon litter treated with one or
more treatment composition components at 50 mM

| a | b | c | f | i | l |
|---|---|---|---|---|---|
| HCl | Acetic Acid | 50 mM Fe | 50 mM Fe | 50 mM Fe | 50 mM |
| | | 50 mM | 50 mM | | citrate |
| | | citrate | citrate | | |
| | | .05% | | | |
| | | chitosan | | | |
| Growth | Growth | Slight | Moderate | Growth | Growth |
| | | Growth | Growth | | |

TABLE 5B
Growth of Salmonella upon litter treated with one or
more treatment composition components at 40 mM

| d | g | j | m | | |
|---|---|---|---|---|---|
| 40 mM Fe | 40 mM Fe | 40 mM Fe | 40 mM | NB control | Growth |
| 40 mM | 40 mM | | citrate | No cells | Control |
| citrate | citrate | | | No litter | |
| .04% | | | | | |
| chitosan | | | | | |
| Moderate | Moderate | Growth | Growth | No Growth | Growth |
| Growth | Growth | | | | |

TABLE 5C
Growth of Salmonella upon litter treated with one or
more treatment composition components at 30 mM

| e | h | k | n |
|---|---|---|---|
| 30 mM Fe | 30 mM Fe | 30 mM Fe | 30 mM citrate |
| 30 mM citrate | 30 mM citrate | | |
| .03% chitosan | | | |
| Moderate Growth | Growth | Growth | Growth |

### Example 5

Assays were performed to examine whether the test strain (Salmonella choleraesuis) was capable of growth at the pHs observed in the tubes in Example 4. The pH of one 18 mL aliquot of NB was adjusted with HCl to pH 4.8 (tube c). In addition, tubes were prepared using litter treated with either 100 mM Fe+100 mM Citrate+0.1% chitosan (tube a) or 50 mM Fe+50 mM Citrate+0.05% chitosan (tube b). The tubes were inoculated with the same number of cells as in the previous experiments and incubated at 37° C. for 24 hours. The results from these tests are shown in Table 6.

The test strain grew at low pH after 24 hours of incubation. This suggests that the acidic environment created by our treated litter is not the sole cause of its germicidal activity. The litter used in tubes a and b of this experiment had been prepared for the previous experiments, thus providing confirmation that 1) litter treated with 50 mM ferric chloride+50 mM citrate+0.05% chitosan was effective as a microbial growth inhibitor, and 2) treated litter remains effective after long periods of storage (at least 2 weeks, in this example).

TABLE 6
Growth of Salmonella upon exposure to
Treated Litter versus pH 4.8 medium

| a | b | c |
|---|---|---|
| 100 mM Fe | 50 mM Fe | nutrient broth |
| 100 mM Citrate | 50 mM Citrate | pH 4.8 |
| .1% chitosan | .05% chitosan | |
| (prepared May. 30, 2001) | (prepared Jun. 5, 2001) | |
| No Growth | No Growth | Growth |

### Example 6

The litter prepared in Example 4 was re-tested for inhibition of bacterial growth in this example, following the same protocols as used in the previous examples. As in the previous experiments, 0.5 g of each litter of the following litter types was added to capped test tubes (in duplicate).

a. 50 mM Fe+50 mM Citrate+0.05% chitosan

b. 40 mM Fe+40 mM Citrate+0.04% chitosan

c. 30 mM Fe+30 mM Citrate+0.03% chitosan

d. 50 mM Fe

e. 50 mM Citrate

Two extra tubes were used for positive growth (cells only) and negative growth (no cells) growth controls. 18 ml of NB was added to each tube, and all tubes (except the “no cells” control tubes). The tubes were incubated for 24 hours at 37° C., after which the pH was measured and the number of tubes for each treatment showing signs of bacterial growth were counted. The results are provided in Table 7.

As in the previous experiments, both replicates of each test showed identical results. Litter treated with 50 mM iron was slightly darker than the control, while the treatment compositions having lower iron concentrations showed very little or no color change.

The controls showed the expected results (e.g., growth in the positive control, no growth in the negative control). The 0.03% chitosan treated litter showed slight growth under careful visual inspection. However, the solution did not become noticeably turbid, even after several days. The litter treated with 40 mM Fe+40 mM Cit+0.04% chitosan showed even less growth. Higher concentrations appeared to be completely inhibitory.

As expected, the litter treated with 50 mM FeCl₃+50 mM Citrate+0.05% chitosan was effective against all strains present. Though lower concentrations of the treatment composition were not completely inhibitory, very little growth was observed (even after several days of incubation). Thus, lower concentrations of iron, citrate, and chitosan are also effective.

The pH of the litter-containing solutions decreased with increasing treatment concentration. Though the acidic environments may be having some inhibitory effects, they are clearly not the primary reason for the germicidal activity of the treated litter.

TABLE 7
Growth of Salmonella upon litter treated with
various concentrations of treatment composition

| a | b | c | d | e | f | g |
|---|---|---|---|---|---|---|
| 50 mM Fe | 30 mM Fe | 40 mM Fe | 50 mM | 50 mM Fe | NB | Growth |
| 50 mM Cit | 30 mM Cit | 40 mM Cit | Cit | | Control | Control |
| .05% | .03% | .04% | | | No Cells | NB + Cells |
| chitosan | chitosan | chitosan | | | | |
| No | Slight | Very | Growth | Growth | No | Growth |
| Growth | Growth | Slight | | | Growth | |
| | | Growth | | | | |
| pH 4.7 | pH 5.3 | pH 4.9 | pH 6.4 | pH 6.3 | pH 6.7 | pH 6.6 |

### Example 7

Treatment compositions containing an iodophore (in this example, povidone-iodine complex, or “PIC”) were tested for germicidal activity. Litter was prepared using the protocol described in the previous examples. Approximately 7 g of litter was treated with one of the following solutions for 30 minutes:

(a) 20 mM FeCl₃+20 mM Citrate+0.02% chitosan+0.1% PIC

(b) 10 mM FeCl₃+10 mM Citrate+0.01% chitosan+0.1% PIC

(c) 0.25% PIC

The litter was dried, then approximately 0.5 g litter was added to tubes (in duplicate) containing nutrient broth and inoculated with Salmonella. In addition, two controls containing only nutrient broth (no litter) were prepared; one control tube was inoculated with Salmonella to serve as the growth control. The tubes were incubated overnight (24 hrs) at 37° C., and examined for bacterial growth. The results are shown in Table 8.

Both concentrations of the iron-containing composition inhibited bacterial growth, and the solutions did not discolor after soaking of the litter. The pH of the solution from the 10 mM FeCl₃-containing tube was 5.4, while the pH of the solution from the 20 mM FeCl₃-containing tube was 5.3.

The addition of PIC dramatically increased the efficacy of the treated litter. Prior experiments indicated that litter treated with 10 mM Fe+10 mM Citrate+0.01% chitosan was only slightly inhibitory. Though 0.1% PIC was ineffective alone, litter treated with 10 mM Fe+10 mM Citrate+0.01% chitosan+0.1% PIC was completely effective. Furthermore, the pH of the solution in this tube was 5.4. This pH does not inhibit the growth of the test organism, confirming that the acidic environment is not responsible for the observed germicidal activity in this series of experiments.

TABLE 8
Growth of Salmonella in the Presence of an Iodophore

| a | b | c | d | e |
|---|---|---|---|---|
| 20 mM Fe | 10 mM Fe | .25% PIC | Growth | NB |
| 20 mM Cit | 10 mM Cit | | Control | Control |
| .02% chitosan | .01% chitosan | | | |
| .1% PIC | .1% PIC | | | |
| No Growth | No Growth | Growth | Growth | No Growth |

### Example 8

Several of the litter preparations described previously were tested for their ability to inhibit the growth of Salmonella-inoculated chicken feces. The litter preparations to be tested were:

(1) 40 mM FeCl₃+40 mM Citrate+0.04% chitosan+0.1% PIC

(2) 100 mM FeCl₃+100 mM Citrate+0.1% chitosan

(3) dH₂O (Control)

The Salmonella cultures and litter preparations were prepared as described in the previous experiments. The chicken feces were prepared as follows. Dry chicken feces were autoclaved for 35 minutes, then 3 g aliquots of the sterile feces were inoculated with 5 mL of 10⁴ cfu/ml Salmonella culture. The inoculated feces were mixed using a stomacher (Seward Stomacher 80 Lab System, from Seward Ltd., London, UK) for approximately 30 seconds. Approximately 1 g of litter preparation was added to each stomacher bag containing inoculated feces aliquots, and the mixtures were further processed for about 60 seconds. The mixtures were then placed in separate weigh boats and allowed to dry overnight.

Approximately 0.5 g of the dried mixture was added to 10 mL nutrient broth in a stomacher bag, and the reconstituted mixture was processed for 60 seconds in the stomacher device. 100 μl of liquid sample (preferably taken from the middle of the solution immediately after stomaching) was added to 900 μL nutrient broth in a snap-cap Eppendorf tube. Serial dilutions ( through 10⁻⁵ the starting concentration) were generated, vortexed thoroughly, and plated (200 μL) onto MacConkey agar. Alternatively, the serial dilutions are plated onto BG Sulfa agar for selective analysis of Salmonella growth inhibition. The plated were incubated for 1 day, and the resulting colonies counted. The results are shown in Table 9.

TABLE 9
Growth of Salmonella-inoculated Chicken Feces on Treated Litter

| | Number of | | | | | | | | | | |
|---|---|---|---|---|---|---|---|---|---|---|---|
| | Colonies | | | | | CFU/g of Dried Litter Mixture | | | | | Average |
| Dilutions -> | 10⁻¹ | 10⁻² | 10⁻³ | 10⁻⁴ | 10⁻⁵ | 10⁻¹ | 10⁻² | 10⁻³ | 10⁻⁴ | 10⁻⁵ | CFU/g |
| Control (H₂O) | 124 | 9 | 5 | 0 | 0 | 124000 | 90000 | 500000 | | | 2.38E+05 |
| 100 mM Fe | 0 | 0 | 0 | 0 | 0 | | | | | | No |
| 100 mM Cit | | | | | | | | | | | Salmonella |
| .1% chitosan | | | | | | | | | | | detected. |
| 40 mM Fe | 0 | 0 | 0 | 0 | 0 | | | | | | No |
| 40 mM Cit | | | | | | | | | | | Salmonella |
| .04% chitosan | | | | | | | | | | | detected. |
| .1% PlC | | | | | | | | | | | |

Number of Colonies) * 5 * (Dilution Factor) = CFU/ml,
CFU/ml * 10ml = CFU/.5 g,
(CFU/.5 g) * 2 = CFU/g

Kits

In an additional aspect, the present invention provides kits embodying the methods and compositions for mitigation of microbial contamination in animal litter and/or animal containment areas, as described herein. The kits optionally comprise one or more of a) containers for packaging one or more composition elements, b) sponges, cloths, trays, pumps, spraying devices or other devices for contacting the animal litter or containment area with the compositions of the present invention, c) aqueous solutions for use with the composition, d) packaging materials, and the like. Furthermore, instructions, such as written directions or videotaped demonstrations detailing the use of the kits of the present invention, i.e., according to the methods set forth herein, are optionally provided with the kit.

In a further aspect, the present invention provides for the use of any composition or kit herein, for the practice of any method or assay herein, and/or for the use of any apparatus or kit to practice any assay or method herein.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

What is claimed is:

1. An antimicrobial composition for treating a quantity of animal litter, the composition comprising:
a preparation of at least one iron salt;
a citrate composition; and
a chitosan preparation.

2. The composition of claim 1, wherein the at least one iron salt comprises FeCl₃.

3. The composition of claim 1, wherein the citrate composition comprises sodium citrate.

4. The composition of claim 1, wherein the chitosan preparation comprises low molecular weight chitosan.

5. The composition of claim 1, wherein the composition comprises an aqueous treatment solution comprising about 100 mM FeCl₃, about 100 mM citrate, and about 0.1% chitosan.

6. The composition of claim 1, wherein the composition comprises an aqueous treatment solution comprising about 50 mM FeCl₃, about 50 mM citrate, and about 0.05% chitosan.

7. The composition of claim 1, wherein the composition comprises a powdered preparation comprising about 46% (w/w) FeCl₃, about 54% (w/w) citrate.

8. The composition of claim 1, further comprising an iodophore.

9. The composition of claim 8, wherein the iodophore comprises a povidone-iodine complex.

10. A method for preparing antimicrobial animal litter, the method comprising:
a) providing a first quantity of animal litter;
b) providing the composition of claim 1; and
c) exposing the first quantity of animal litter to the composition of claim 1, thereby preparing an antimicrobial animal litter.

11. The method of claim 10, wherein providing the first quantity of animal litter comprises providing one or more litter components selected from the group consisting of rice hulls, straw, corn husks, clay, diatomaceous earth; sawdust; wood chips, wood shavings, recycled paper products; agricultural waste materials, and gravel.

12. The method of claim 10, wherein providing the composition of claim 1 comprises providing a powdered treatment preparation comprising about 45% FeCl₃, about 54% citrate, and about 0.001% chitosan; and wherein exposing the first quantity of animal litter to the composition comprised coating the animal litter with the powdered treatment composition.

13. The method of claim 10, wherein providing the composition of claim 1 comprises providing an aqueous treatment preparation and wherein exposing the first quantity of animal litter comprises soaking the animal litter in the aqueous treatment solution.

14. The method of claim 10, wherein providing the composition of claim 1 comprises providing an aqueous treatment preparation and wherein exposing the first quantity of animal litter comprises spraying the animal litter with the aqueous treatment solution.

15. The method of claim 10, wherein providing the composition of claim 1 comprises preparing an aqueous treatment solution of about 100 mM FeCl₃, about 100 mM citrate, and about 0.1% chitosan.

16. The method of claim 15, wherein preparing the aqueous solution further comprises suspending the chitosan in an organic acid prior to combining the chitosan with the FeCl₃ and citrate.

17. The method of claim 16, wherein the organic acid comprises one or more of citric acid, acetic acid, or lactic acid.

18. The method of claim 10, wherein providing the composition of claim 1 comprises preparing an aqueous treatment solution of about 50 mM FeCl₃, about 50 mM citrate, and about 0.05% chitosan.

19. The method of claim 10, wherein exposing the first quantity of animal litter to the treatment solution further comprises removing an unabsorbed portion of the treatment solution from the animal litter.

20. The method of claim 10, further comprising:
combining a second quantity of animal litter with the treated animal litter, wherein the second quantity of animal litter is about equal to the first quantity of animal litter.

21. A treated animal litter composition prepared by the method of claim 10.

22. The composition of claim 21, wherein the animal litter comprises one or more components selected from the group consisting of rice hulls, straw, corn husks, clay, diatomaceous earth; sawdust; wood chips, wood shavings, recycled paper products; agricultural waste materials, and gravel.

23. The composition of claim 21, wherein the animal litter comprises chicken litter.

24. The composition of claim 21, wherein the composition further comprises a second quantity of animal litter, wherein the second quantity of animal litter is about equal to the first quantity of animal litter.

25. A method for reducing the microbial population in an animal containment area, the method comprising treating the animal containment area with the treated animal litter composition of claim 21.

26. A method for increasing the storage life of an animal litter, the method comprising treating the animal litter with the composition of claim 1.
